(19) United States
(12) Patent Application Publication
(10) Pub. No.: US 2002/0048593 A1
(43) Pub. Date: Apr. 25, 2002

(54) Use of phytanetriol as an anti-pollution agent, in particular in a cosmetic composition

(75) Inventor: Bruno Biatry, Vincennes (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'Oreal, Paris (FR)

(21) Appl. No.: 09/875,994

(22) Filed: Jun. 8, 2001

(30) Foreign Application Priority Data
Jun. 8, 2000 (FR) 0007343

(51) Int. Cl. A61K 7/00

(52) U.S. Cl. 424/401

(57) ABSTRACT

The present application relates to the use in topical application of phytanetriol as an antipollution agent, in particular as an anti-pollution cosmetic agent. The application also relates to a cosmetic treatment process for protecting the body against the effects of pollution, which includes applying to the keratin material a composition containing an effective amount of phytanetriol in a physiologically acceptable medium. Phytanetriol (3,7,1 1,15-tetramethyl-1,2,3-hexadecanetriol) is preferably in an O/W or W/O emulsion, or in the form of cubic gel particles.

### BACKGROUND OF THE INVENTION

1. Field of the Invention

The present application relates to the use in topical applications of phytanetriol as an antipollution agent, in particular as an anti-pollution cosmetic agent, and to a cosmetic treatment process for protecting the body against the effects of pollution, which includes applying to the keratin material a composition containing an effective amount of phytanetriol in a physiologically acceptable medium.

2. Discussion of the Background

Certain urban environments are regularly subjected to peaks of pollution. An individual in his daily environment, and particularly in an urban area, may be subjected to a whole range of factors attacking keratin materials, and in particular, the skin, the scalp and the hair, by various airborne pollutants.

Among the pollutants which may exert deleterious effects on keratin materials, toxic gases such as ozone, carbon monoxide, nitrogen oxides or sulphur oxides are among the major constituents. It has been found that these toxic gases promote the desquamation of keratin materials, making them dull and dirty. Similarly, these gases cause cellular asphyxia of the said keratin materials.

It is moreover known that heavy metals (lead, cadmium and mercury) are atmospheric pollutants whose emissions have increased considerably, especially in urban and industrial environments. In addition to certain toxic effects which they cause, heavy metals have the property of reducing the activity of the cellular defence means against free radicals (see for example R. S. Dwivedi, J. Toxicol. Cut. & Ocular Toxical. 6(3); 183-191 (1987)). Thus, heavy metals aggravate the toxic effects of gaseous pollutants by reducing the efficacy of the natural defence means, and bring about an acceleration of the phenomenon of cell aging. This is particularly true for keratin materials and especially the skin, the scalp and the hair, which are in direct and permanent contact with the external environment.

Thus, the harmful effects of pollution on keratin materials affect cell respiration and are reflected by accelerated aging of the skin, with a dull complexion and the early formation of wrinkles or fine lines, and also by a reduction in the vigour of the hair, which thus acquires a dull appearance. Dehydration of the skin is also observed as a harmful effect of pollution. In addition, pollution causes an increase in the flow of sebum, the consequence of which is that the skin and the hair become dirty more quickly. Furthermore, pollution may cause allergy and irritation phenomena on the skin.

Thus, there is a need for compositions to prevent the harmful effects due to pollutants (gases or heavy metals), so as to protect keratin materials against these external pollutants.

Phytanetriol, or 3,7,11,15-tetramethyl-1,2,3-hexadecanetriol, is a known compound and is sold in particular under the name “Phytanetriol-63926” by the company Roche. Admittedly, it is known practice to use phytanetriol in topical application to care for the skin (see GB-A-923 400 and EP-A-0 584 315), as a moisturizer (see GB-A-2 304 573 and EP-A-0 343 444) and as a penetration modifier (see documents EP-A-0 579 079 and GB-A-2 304 573). Its use is also known in the field of haircare (see 15 GB-A-944 834, U.S. Pat. No. 5,776,443 and WO-A-00/15181) and in make-up (see FR-A-2 675 045 and U.S. Pat. No. 5,102,654). However, no document discloses that phytanetriol can have properties of protecting keratin materials against pollution.

### SUMMARY OF THE INVENTION

One object of the present invention is to protect keratin materials against the harmful effects due to pollutants (gases or heavy metals).

Another object of the present invention is to prevent the harmful effects on keratin materials due to pollutants (gases or heavy metals).

It has now been found, entirely surprisingly, that phytanetriol protects keratin materials against the effects of pollutants found in the atmosphere.

Thus, one subject of the present invention relates to the cosmetic use of phytanetriol as an anti-pollution agent in a composition for topical application to keratin materials.

Another embodiment of the invention is also the use of phytanetriol to prepare a topical-application composition for protecting keratin materials against the harmful effects of pollution.

Another embodiment of the present invention provides a method of protecting keratin materials from the harmful effects of pollution, including topically applying a composition containing an effective amount of phytanetriol to the keratin materials.

Another embodiment of the present invention provides a treatment process for protecting a keratin material against the effects of pollution, including applying to the keratin material a composition containing an effective amount of phytanetriol in a physiologically acceptable medium.

Another embodiment of the present invention provides a treatment process for improving the cell respiration and/or for reducing the desquamation of a keratin material and/or for preventing a keratin material from becoming dull and/or dirty and/or for preventing the dehydration of a keratin material, including applying to the keratin material a composition containing an effective amount of phytanetriol in a physiologically acceptable medium.

Another embodiment of the present invention provides a process for treating dry skin, including applying to the skin a composition containing an effective amount of phytanetriol in a physiologically acceptable medium.

Another embodiment of the present invention provides a composition, including:

phytanetriol in the form of cubic gel particles,

wherein said cubic gel particles are formed from a mixture including:

(i) 0.1% to 15% by weight, relative to the total weight of the composition, of phytanetriol or of a mixture of phytanetriol with a compound selected from the group including N-2-alkoxycarbonyl derivatives of N-methylglucamine and unsaturated fatty acid monoglycerides; and

(ii) 0.05 to 3% by weight, relative to the total weight of the composition, of at least one dispersing and stabilizing agent, said agent being selected from the group including surfactants that are water-soluble at room temperature and containing a saturated or unsaturated, linear or branched fatty chain containing from 8 to 22 carbon atoms.

### BRIEF DESCRIPTION OF THE PREFERRED EMBODIMENTS

Various other objects, features and attendant advantages of the present invention will be more fully appreciated as the same becomes better understood from the following detailed description of the preferred embodiments of the invention.

The expression “topical application” means herein an external application to keratin materials, these especially being the skin, the scalp, the eyelashes, the eyebrows, the nails and mucous membranes.

The phytanetriol may be present in the composition for topical application in an amount ranging, for example, from 0.001% to 20% by weight and preferably from 0.1% to 10% by weight relative to the total weight of the composition. These ranges include all values and subranges therebetween, including 0.01, 0.05, 0.5, 1, 2, 3, 4, 5, 6, 7, 8, 9, 11, 12, 13, 14, 15, 16, 17, 18, and 19%.

The phytanetriol used according to the invention may be incorporated especially into compositions which may in particular be in the form of oil-in-water (0/W) or water-in-oil (W/0) emulsions or in the form of cubic gel particles, these particles possibly being used alone or incorporated in an emulsion.

The term “cubic gel” denotes transparent gels which are isotropic in polarized light and which are in the form of a cubic liquid crystal phase. The cubic phases are organized in a bipolar manner into distinct hydrophilic and lipophilic domains, in close contact and forming a thermodynamically stable three-dimensional network. Such an organization has been disclosed in particular in Luzzati (1968), “Biological Membranes” (Chapman, D. Ed.), vol. 1, 71-123 and in Mariani et al. (1988), J. Mol. Biol., 204, 165-189, and also in “La Recherche” (1992), vol. 23, 306-315, the entire contents of each of which being hereby incorporated by reference. According to the arrangement of the hydrophilic and lipophilic domains, the cubic phase is said to be of normal or inverse type. The term “cubic gel” used according to the present invention combines, of course, gels with cubic phases of different types. Any type of cubic gel may be used according to the present invention.

When cubic gel is dispersed in aqueous medium, cubic gel particles in dispersion are obtained, particles which have the same structure as cubic gel.

The cubic gel particles containing phytanetriol may be present in the topical-application composition used according to the invention in an amount ranging, for example, from 0.1% to 20% by weight and preferably from 0.1% to 10% by weight relative to the total weight of the composition. These ranges include all values and subranges therebetween, including 2, 3, 4, 5, 6, 7, 8, 9, 11, 12, 13, 14, 15, 16, 17, 18, and 19%.

The cubic gel particles containing phytanetriol are advantageously in aqueous dispersion in the topical-application composition. These particles may be obtained in particular by the preferred embodiment described below.

According to this embodiment, the particles are in aqueous dispersion and are formed from a mixture including (i) 0.1% to 15% by weight, relative to the total weight of the composition, of phytanetriol or of a mixture of phytanetriol with a compound chosen from N-2-alkoxycarbonyl derivatives of N-methylglucamine and unsaturated fatty acid monoglycerides, and (ii) 0.05 to 3% by weight, relative to the total weight of the composition, of at least one dispersing and stabilizing agent, the said agent being chosen from surfactants that are water-soluble at room temperature, containing a saturated or unsaturated, linear or branched fatty chain containing from 8 to 22 carbon atoms. The percentages are expressed herein relative to the total amount of the composition containing the phytanetriol-based cubic gel particles. The range for (i) includes all values and subranges therebetween, including 0.5, 1, 2, 3, 4, 5, 6, 7, 8, 9, 10, 11, 12, 13, and 14%. The range for (ii) includes all values and subranges therebetween, including 0.1, 1.1, 1.5, 2, 2.5 and 2.75%.

According to this embodiment of the cubic gel particles used according to the invention, the relative weight proportion of phytanetriol relative to the weight of the dispersing and stabilizing agent (ii) may range, for example, from 2 to 200 and is preferably less than or equal to 50. These ranges include all values and subranges therebetween, including 3, 5, 10, 20, 30, 40, 75, 100, 125 and 150.

Among the N-2-alkoxycarbonyl derivatives of N-methylglucamine which may be used as a mixture with phytanetriol, mention may be made in particular of those corresponding to formula (I) below:

in which R represents a branched alkyl radical containing from 6 to 18 carbon atoms.

Among these derivatives, mention may be made in particular of N-2-hexyldecyloxycarbonyl-

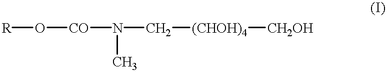

N-methyl-glucamine, N-2-ethylhexyloxycarbonyl-N-methylglucamine and N-2-butyloctyloxycarbonyl-N-methylglucamine, and mixtures thereof.

The compounds of formula (I) as defined above are disclosed and may be prepared according to the process disclosed in EP-A-711 540, which is hereby incorporated in its entirety by reference. This process in particular includes the steps:

(a) dissolving N-methylglucamine in a mixture of water and an organic solvent, the solvent preferably being tetrahydrofuran, for example,

(b) dispersing sodium bicarbonate in the mixture obtained above, in a suitable proportion corresponding to about four times the molar proportion of N-methylglucamine,

(c) then introducing an alkyl chloroformate, the alkyl radical being C₆-C₁₈, into the reaction mixture obtained, in a suitable proportion, generally an equimolar proportion relative to that of N-methylglucamine, and then leaving the mixture to react, and

(d) filtering the reaction mixture obtained after step (c), collecting the pasty residue obtained by filtration and then dissolving it in acetone to crystallize it at a temperature of about 5° C.

After filtration, the crystals of the N-2-alkoxycarbonyl derivative of N-methylglucamine formed are spin-filtered and dried under vacuum.

When phytanetriol is used as a mixture with one or more compounds of formula (I), this mixture preferably includes an amount of phytanetriol ranging from 1% to 40% by weight and better still from 10% to 30% by weight relative to the weight of the mixture, and an amount of N-2-alkoxycarbonyl derivative of N-methylglucamine of formula (I) ranging from 60% to 99% by weight and better still from 70% to 90% by weight relative to the weight of the mixture. These ranges include all values and subranges therebetween, including 2, 5, 7, 15, 20, 25, 35, 50, 55, 65, 75, 80, 85, and 95% as appropriate for the respective amounts of phytanetriol and N-2-alkoxycarbonyl derivative of N-methylglucamine of formula (I).

The unsaturated fatty acid monoglycerides which may be used as a mixture with phytanetriol to prepare cubic gel particles are preferably those with an unsaturated fatty chain containing from 16 to 22 carbon atoms. Among these monoglycerides, mention may be made in particular of glyceryl monooleate or monooleine and glyceryl monolinoleate or monolinoleine. Needless to say, to prepare the dispersions of cubic gel particles, it is possible to use a mixture of monoglycerides as defined above, and also a mixture of unsaturated fatty acid monoglycerides and of saturated fatty acid monoglycerides, the proportion of saturated fatty acid monoglycerides however preferably being less than that of the unsaturated fatty acid monoglycerides.

When phytanetriol is used as a mixture with unsaturated fatty acid monoglycerides, this mixture preferably includes an amount of phytanetriol ranging from 1% to 50% by weight and better still from 10% to 30% by weight relative to the total weight of the mixture and an amount of unsaturated fatty acid monoglyceride in a proportion of from 50% to 99% by weight and better still from 70% to 90% by weight relative to the weight of the mixture. These ranges include all values and subranges therebetween, including 2, 5, 7, 15, 20, 25, 35, 55, 65, 75, 80, 85, and 95% for the respective amounts of phytanetriol and unsaturated fatty acid monoglyceride as appropriate.

The agent (ii) for dispersing and stabilizing the cubic gel particles is preferably chosen from:

(1) alkyl or alkenyl ethers or esters of a polyol,

(2) N-acyl amino acids and derivatives thereof, and peptides N-acylated with an alkyl or alkenyl radical, and salts thereof,

(3) alkyl or alkenyl ether or ester sulphates, derivatives thereof and salts thereof,

(4) polyoxyethylenated fatty alkyl or alkenyl ethers or esters,

(5) polyoxyethylenated alkyl or alkenyl carboxylic acids and salts thereof,

(6) N-alkyl or alkenyl betaines,

(7) alkyl or alkenyl trimethylammoniums and salts thereof, and

(8) mixtures thereof.

In the compounds listed above, the alkyl and alkenyl radicals contain from 8 to 22 carbon atoms and may be in the form of mixtures.

(1) As alkyl or alkenyl ethers or esters of a polyol, mention may be made in particular of:

(a) sorbitan alkyl or alkenyl esters polyoxyethylenated with at least 20 ethylene oxide units, such as sorbitan palmitate 20 EO or Polysorbate 40 sold under the name “Montanox 40 DF” by the company SEPPIC, and sorbitan laurate 20 EO or Polysorbate 20 sold under the name “Tween 20” by the company ICI,

(b) oxyethylenated or non-oxyethylenated polyglyceryl alkyl or alkenyl esters including at least 10 units derived from glycerol, such as polyglyceryl-10 laurate sold under the name “Decaglyn 1-L” by the company Nikko Chemicals,

(c) polyglyceryl alkyl or alkenyl ethers, such as polyglyceryl-3 hydroxylauryl ether sold under the name “Chimexane NF” by the company Chimex, and

(d) alkyl or alkenyl esters or ethers of mono- or polysaccharides, such as those derived from glucose, fructose, galactose, maltose or lactose, and in particular 1- and 6- monoesters of D-fructose, of decylglucose and of decylpolyglucose.

(2) As N-acyl amino acids and derivatives thereof, and peptides N-acylated with an alkyl or alkenyl radical, and salts thereof, the ones that are preferably used are those for which the alkyl or alkenyl radical contains at least 12 carbon atoms.

According to the invention, the term “amino acids” means alpha-, beta- or gamma-amino acids. N-acyl amino acid salts which may be mentioned, for example, are those of N-acylglutamate, such as monosodium cocoylglutamate, monosodium lauroylglutamate, disodium (C₁₄-C₂₀) alkylglutamate (the C₁₄-C₂₀ alkyl radical being derived from hydrogenated tallow), sold respectively under the names “Acylglutamate CS-11”, “Acylglutamate LS-11” and “Acylglutamate HS-21” by the company Ajinomoto. Mention may also be made of N-aryl lysines such as lauroyllysine sold under the name “Amihope LL” by the company Ajinomoto. The N-acyl amino acid derivatives and salts thereof are preferably N-acyl sarcosinates such as the sodium lauroylsarcosinate sold under the name “Oramix L30” by the company SEPPIC and the sodium myristoylsarcosinate and sodium palmitoylsarcosinate sold respectively under the names “Nikkol Sarcosinate MN” and “Nikkol Sarcosinate PN” by the company Nikko Chemicals.

Among the N-acyl peptides which may be mentioned are those derived from all or part of collagen or keratin, such as the sodium lauroyl collagen and palmitoyl keratin sold under the names “Proteol B 30” and “Lipacide PK” by the company SEPPIC.

(3) Among the alkyl or alkenyl ether or ester sulphates, derivatives thereof and salts thereof, the ones that are preferably used are those for which the alkyl or alkenyl radical contains at least 12 carbon atoms.

Among the alkyl or alkenyl ether sulphates, the ones that are preferably used are alkyl ether sulphate salts and in particular sodium lauryl ether sulphate. Among the alkyl or alkenyl ester sulphates which may be mentioned, for example, are isethionic acid esters and its salts, and in particular the sodium cocoyl isethionate sold under the name “Geropon AC 78” by the company Rhone-Poulenc.

(4) Among the polyoxyethylenated fatty alkyl or alkenyl ethers or esters which are preferably used are those for which the alkyl or alkenyl radical contains at least 12 carbon atoms. Those particularly preferred contain at least 20 ethylene oxide units, such as, for example, PEG-20 stearate, laureth-23, oleth-20 and PEG-25 phytosterol.

(5) Among the polyoxyethylenated alkyl or alkenyl carboxylic acids and salts thereof which are preferably used are those including at least 10 ethylene oxide units, such as, for example, laureth-10 carboxylic acid and oleth-10 carboxylic acid.

(6) Among the N-alkyl or alkenyl betaines which are preferably used are those for which the alkyl or alkenyl radical contains at least 12 carbon atoms, such as, for example, laurylamidopropylbetaine and oleylamidopropylbetaine.

(7) Among the alkyl or alkenyl trimethylammoniums and salts thereof which are preferably used are those for which the alkyl or alkenyl radical contains at least 12 carbon atoms. Salts which are preferably used are the bromides and chlorides, such as cocoyltrimethylammonium chloride and cetyltrimethylammonium bromide.

According to one particular embodiment of the invention, a water-insoluble ionic amphiphilic lipid may be added to the aqueous dispersion containing these particles, preferably in an amount ranging from 0.0005 to 5% by weight and better still from 0.001 to 2% by weight relative to the total weight of the dispersion. These ranges include all values and subranges therebetween, including 0.005, 0.01, 0.05, 0.1, 1.1, 1.5, 2.1, 2.5, 3, 3.5 and 4%.

Among the water-insoluble ionic amphiphilic lipids which may be mentioned in particular are:

(i) phospholipids such as natural phospholipids, for instance soya lecithin or egg lecithin, chemically or enzymatically modified phospholipids, for instance hydrogenated lecithin or the sodium salt of phosphatidic acid, and synthetic phospholipids such as dipalmitoylphosphatidylcholine,

(ii) phosphoric esters of fatty acids and salts thereof, in particular the sodium and potassium salts thereof, such as the monocetyl phosphate sold under the name “Monafax 160” by the company Mona, and the dimyristyl phosphate sold under the name “Mexoryl SY” by the company Chimex,

(iii) N-aryl derivatives of glutamic acid and salts thereof, such as the monosodium stearoylglutamate sold under the name “Acylglutamate HS 11” by the company Ajinomoto, and the mixture monosodium cocoyl(C₁₄-C₂₀) alkyl glutamate, the C₁₄-C₂₀ alkyl radical being derived from hydrogenated tallow, sold under the name “Acylglutamate GS 11” by the company Ajinomoto,

(iv) the sodium cetyl sulphate sold under the name “Nikkol SCS” by the company Nikko Chemicals,

(v) the sodium cocoyl monoglyceride sulphate sold under the name “Nikkol SGC 80 N” by the company Nikko Chemicals, and

(vi) water-insoluble quaternary ammonium derivatives such as behenyltrimethylammonium chloride, dilauryldimethylammonium chloride, distearyldimethylammonium chloride, 4, 5-dihydro-1-methyl-2- (C₁₄-C₂₀) alkyl-1-(2-(C₁₄-C₂₀)alkylaminoethyl) imidazolium methyl sulphate, the C₁₄-C₂₀ alkyl radicals being derived from hydrogenated tallow, sold under the name “Rewoquat W75H” by the company Rewo Chemische, dialkylhydroxyethylmethylammonium methyl sulphate whose alkyl radicals are derived from hydrogenated or unhydrogenated tallow, sold under the name “Stepanquat VP 85” by the company Stepan, and “Quaternium-82” sold by the company SEPPIC under the name “Amonyl DM”.

The incorporation of these water-insoluble ionic amphiphilic lipids gives the cubic gel particles a surface charge which results in electrostatic repulsion between the particles.

The cubic gel particles as defined above have a size which may be modified by the nature and concentration of the compounds of which they are made. These particles generally have a number-average size, measured using a BI 90 laser granulometer from the company Brookhaven Instruments Corporation, of about from 0.05 μm to about 1 μm and preferably less than or equal to 0.5 μm. These ranges include all values and subranges therebetween, including 0.075, 0.1, and 0.75 μm.

It is also possible to incorporate active compounds of various types into the cubic gel particles. In particular, the said particles may contain a hydrophilic or lipophilic active principle. Needless to say, by virtue of the specific structure of the cubic gel particles, it is possible to incorporate therein both hydrophilic and lipophilic active principles, even if these active principles are incompatible to a certain extent.

The cubic gel dispersions containing phytanetriol may preferably be obtained according to a preparation process including at least two steps. The first step generally includes preparing an aqueous dispersion of cubic gel particles as defined above, by fragmentation, using a homogenizer, of a cubic gel composed as defined above and of water, optionally in the presence of water-insoluble ionic amphiphilic lipids and/or of hydrophilic and/or lipophilic active principles and/or of a dispersing and stabilizing agent, as are defined above. The homogenizer may be of the rotor-stator type with a high shear rate, such as Virtis® or Heidolph Diax 600®, or a high-pressure homogenizer working at between 200 and 1,800 bar approximately (20 to 180 MPa).

Needless to say, it is possible at this stage in the preparation of the aqueous dispersion of cubic gel particles to introduce various additives and/or active principles into the aqueous phase. After formation of the cubic gel particles, the dispersing and stabilizing agent is generally outside the said particles.

The second step then generally includes adding to the said dispersion obtained an oily phase optionally containing certain lipophilic active principles and/or additives and in subjecting the mixture to a mechanical stirring which may be carried out in particular using a homogenizer of the same type as those defined above.

Various additives and/or active principles may also be introduced at this stage in the preparation. Moreover, when it is desired to prepare a gelled dispersion, in a third step, an aqueous solution containing a gelling agent is generally added to the mixture obtained after the second step.

The compositions containing phytanetriol, which are used according to the invention, may in particular constitute cosmetic and dermatological compositions. For such an application, they contain a physiologically acceptable medium. The expression “physiologically acceptable medium” means herein a medium which is compatible with the skin, the lips, the scalp, the eyelashes, the eyes and/or the hair. This physiologically acceptable medium may more particularly includes water and optionally of a physiologically acceptable organic solvent chosen, for example, from lower alcohols containing from 1 to 4 carbon atoms, for instance ethanol, isopropanol, propanol or butanol; polyethylene glycols containing from 6 to 80 ethylene oxides; polyols, for instance propylene glycol, isoprene glycol, butylene glycol, glycerol or sorbitol. The physiologically acceptable medium of the composition according to the invention has a pH which is compatible with the skin and which preferably ranges from 3 to 8 and better still from 5 to 7.

According to one preferred embodiment, the compositions used in the present invention also include an oily phase, which especially provides a sensation of comfort and softness when applied to the skin. The amount of oily phase may range, for example, from 2% to 40% by weight and preferably from 5% to 25% by weight relative to the total weight of the composition, the remainder of the composition including the aqueous phase containing or consisting of the aqueous dispersion of cubic gel particles. These ranges include all values and subranges therebetween, including 3, 4, 7, 10, 15, 20, 30, and 35%.

The weight ratio of the amphiphilic compounds constituting the particles of the cubic phase and of the oily phase preferably ranges from 0.02/1 to 1/1 and better still from 0.05/1 to 0.5/1. These ranges include all values and subranges therebetween, including 0.03/1, 0.04/1, 0.1/1, 0.2/1, 0.3/1, and 0.4/1.

The oily phase generally includes at least one oil. As oils which may be used in the invention, mention may be made of mineral oils (liquid petroleum jelly, mineral oil), oils of plant origin (liquid fraction of karite butter, sunflower oil or castor oil), oils of animal origin (perhydrosqualene or lanolin oil), synthetic oils (hydrogenated polyisobutene, isostearyl neopentanoate or isopropyl myristate), non-volatile or volatile silicone oils (cyclomethicones such as cyclopentasiloxane) and fluoro oils (perfluoropolyethers). Fatty substances which may also be used are fatty alcohols, fatty acids and waxes. The oily phase of the emulsion may also contain gums such as silicone gums, resins and waxes.

The composition containing an oily phase may be in the form of a water-in-oil (W/O) or oil-in-water (O/W) emulsion. According to one preferred embodiment, it is in the form of an oil-in-water emulsion.

In a known manner, the cosmetic or dermatological composition of the invention may also contain adjuvants that are common in the cosmetic, pharmaceutical or dermatological field, such as hydrophilic or lipophilic gelling agents, hydrophilic or lipophilic active agents, preserving agents, antioxidants, solvents, fragrances, fillers, screening agents, bactericides, odour absorbers, dyestuffs and salts. The amounts of these various adjuvants are those that are conventionally used in the field under consideration, and, for example, from 0.01 to 10% relative to the total weight of the composition. Depending on their nature, these adjuvants may be introduced into the fatty phase, into the aqueous phase and/or into lipid spherules.

As active agents, the composition may in particular contain other anti-pollution active agents, such as the metallothioneins disclosed in document EP-A-557 042, sphingolipids (see EP-A-O577 718) and any other compound having the property of preventing the harmful effects of pollutants; screening agents such as octocrylene and butylmethoxydibenzoylmethane; moisturizers such as polyols and in particular glycerol. The entire contents of each of these references are hereby incorporated by reference.

Gelling agents which may be mentioned, for example, are cellulose derivatives such as hydroxyethylcellulose and alkylhydroxyethylcelluloses such as cetylhydroxyethylcellulose; algal derivatives such as satiagum; natural gums such as tragacanth or guar gum; synthetic polymers such as carboxyvinyl polymers or copolymers and in particular those sold under the names Carbopol® by the company Goodrich or Synthalen® by the company 3V SA. The proportion of gelling agent preferably ranges from 0.1% to 2% relative to the total weight of the composition.

The compositions used according to the invention may be more or less fluid and may have the appearance of a white or colored cream, an ointment, a milk, a lotion, a serum, a paste or a mousse. They may optionally be applied to the skin in the form of an aerosol. They may also be in solid form and, for example, in the form of a stick.

The compositions used according to the invention may in particular constitute a care product and/or make-up product. They may be used in particular to protect the body and in particular keratin materials against the effects of pollution, and/or to improve cell respiration and/or to reduce the desquamation of keratin materials, and in particular the skin, and/or to prevent the increase in the flow of sebum from the keratin materials and thus to prevent the keratin materials, and in particular the skin, from becoming dull or dirty. They may also be used to prevent the dehydration of the skin.

Thus, another subject of the invention includes a treatment process for protecting a keratin material against the effects of pollution, which includes applying to the keratin material a composition containing an effective amount of phytanetriol in a physiologically acceptable medium.

A subject of the invention is also a treatment process for improving the cell respiration and/or for reducing the desquamation of a keratin material and/or for preventing a keratin material from becoming dull and/or dirty, and/or for preventing the dehydration of a keratin material, which includes in applying to the keratin material a composition containing an effective amount of phytanetriol in a physiologically acceptable medium.

Due to the fact that phytanetriol can prevent dehydration of the skin, it is also suitable for treating dry skin.

Thus, a subject of the invention is also a process for treating dry skin, which includes applying to the skin a composition containing an effective amount of phytanetriol in a physiologically acceptable medium.

The expression “effective amount” means an amount which is sufficient to achieve the desired aim. In practice, this amount may range, for example, as indicated above, from 0.001 to 20% by weight and preferably from 0.1% to 10% by weight relative to the total weight of the composition. These ranges include all values and subranges therebetween, including 0.005, 0.01, 0.05, 0.5, 1, 2, 3, 4, 5, 6, 7, 8, 9, 11, 12, 13, 14, 15, 16, 17, 18, and 19%.

### EXAMPLES

Having generally described this invention, a further understanding can be obtained by reference to certain specific examples which are provided herein for purposes of illustration only and are not intended to be limiting unless otherwise specified. The names are, depending on the case, the chemical names or CTFA (International Cosmetic Ingredient Dictionary and Handbook) names and the amounts are in percentages by weight, except where otherwise mentioned.

### Example 1

| Phase A: | |
|---|---|
| Phytanetriol | 3.92% |
| Cetyl phosphate (sold under the name | 0.08% |
| “Arlatone MAP160” by the company Uniqema) | |
| Water | 1.6% |
| Phase B: | |
| Polysorbate 40 (sold under the name | 1% |
| “Montanox 40 DF” by the company SEPPIC) | |
| (dispersant) | |
| Triethanolamine | 0.04% |
| Water | 55.96% |
| Preserving agent | 0.3% |
| Phase C: | |
| Hydrogenated polyisobutene | 7.8% |
| Cyclohexasiloxane | 11.6% |
| Isostearyl neopentanoate | 2.6% |
| Fragrance | 0.1% |
| Phase D: | |
| Cetylhydroxyethylcellulose (sold under the | 1% |
| name “Natrosol Plus Grade 330CS” by the | |
| company Hercules) | |
| Water | 14% |

Procedure:

First Step:

The compounds of phase A are mixed together at room temperature. Phase B is added to this mixture at room temperature. The mixture is then dispersed and homogenized at room temperature using an “UltraTurrax T50” homogenizer fitted with a 45F dispersion head, at 10 000 rpm for 30 minutes.

Second Step:

The oily mixture of phase C is added to the aqueous dispersion of phytanetriol obtained above. The mixture is then homogenized at room temperature using a high-pressure homogenizer, by 4 homogenization treatments at 600 bar.

Third Step:

The preparation obtained in the second step is gelled using the mixture of phase D. The mixture is then homogenized at room temperature using a paddle homogenizer for 30 minutes. A stable homogeneous cream which can be applied easily to the skin and which protects it against pollution is obtained.

| Example A (comparative): Oil-in-water emulsion | |
|---|---|
| Hydrogenated polyisobutene | 7.8% |
| Cyclohexasiloxane | 11.6% |
| Isostearyl neopentanoate | 2.6% |
| Preserving agents | 0.5% |
| Xanthan gum | 0.6% |
| Polyacrylamide/C13-14 isoparaffin/laureth-7 | 2% |
| (Sepigel 305 sold by the company SEPPIC) | |
| Dimethicone copolyol (DC2-5695, Dow Corning) | 3% |
| Glycerol | 3% |
| Water | qs 100% |
| Example B (comparative): Water-in-oil emulsion | |
| Hydrogenated polyisobutene | 7.8% |
| Cyclohexasiloxane | 11.7% |
| Isostearyl neopentanoate | 2.6% |
| Sodium chloride | 0.6% |
| Cetyldimethicone copolyol (Abil EM90, | 3% |
| Goldschmidt) | |
| Glycerol | 3% |
| Water | qs 100% |

### Test to Demonstrate in vitro the Protective Effect of Phytanetriol Anti-pollution efficacy on Reconstructed Skin

The compositions of Example 1 and of Comparative Examples A and B were applied to the surface of reconstructed-skin epidermal samples (2 mg/cm²) and left in contact with them for 30 minutes at room temperature. Carbon-14 radio labeled particles were then applied to the epidermal samples and left in contact with them for 2 hours in the usual epidermal maintenance medium. Next, the epidermal samples were removed from their maintenance medium and washed several times with PBS buffer (phosphate-buffered saline). The washings allow weakly adsorbed particles to be removed from the epidermal samples without removing the composition initially applied. The levels of residual radio labeled particles were then evaluated by measuring the carbon-14 radioactivity added to the particles. The table below gives the results as percentages of residual particles relative to the amount of particles applied.

| | % relative to the amount |
|---|---|
| | of particles applied |
| Untreated area | 36.2 ± 2.83 |
| Area treated with composition of Example 1 | 3.3 ± 0.66 |
| Area treated with emulsion of Example A | 11.3 ± 0.65 |
| (comparative) | |
| Area treated with emulsion of Example B | 11.2 ± 2.10 |
| (comparative) | |

These results show that the compositions containing phytanetriol which are used according to the invention allow better protection of the skin against pollutant particles than conventional emulsions, by limiting the penetration of the external pollutant particles.

### Example 2: Fluid (O/W Emulsion)

According to the same procedure as for Example 1, a day fluid in the form of a dispersion was prepared by mixing together the following parts:

| Phase A: | |
|---|---|
| Phytanetriol | 2.97% |
| Monosodium stearoylglutamate, sold under | 0.03% |
| the name Acylglutamate HS-11 by the company | |
| Ajinomoto | |
| Water | 1.25% |
| Phase B: | |
| Polysorbate 40 (sold under the name “Montanox | 0.75% |
| 40 DF” by the company SEPPIC) (dispersant) | |
| Glycerol | 4% |
| Propylene glycol | 4% |
| Water | 55.8% |
| Preserving agent | 0.2% |
| Phase C: | |
| Octocrylene | 8.4% |
| Butylmethoxydibenzoylmethane | 3.6% |
| Dimethicone (DC200 Fluid 1.5 cSt) | 4% |
| Isostearyl neopentanoate | 2% |
| Isopropyl myristate | 2% |
| Phase D: | |
| Preserving agent | 1% |
| Water | 10% |

A fluid composition is obtained, which may be applied in the form of a spray and which allows good protection of the skin against pollutants.

### Example 3

Oil-in-water emulsion

| Phase A: | |
|---|---|
| PEG-20 stearate | 4.5% |
| Phytanetriol | 3% |
| Disodium salt of N-stearoylglutamic acid | 0.5% |
| Mineral oil | 10.51% |
| Castor oil | 2.09% |
| Liquid petroleum jelly | 1.94% |
| Isopropyl myristate | 1.39% |
| Lanolin oil | 1.07% |
| Phase B: | |
| Water | 69% |
| Glycerol | 5% |
| Preserving agent | 1% |

A fluid composition which allows good protection of the skin against pollutant particles is obtained.

This application is based on French patent application 0007343, filed Jun. 8, 2000, the entire contents of which are hereby incorporated by reference, the same as if set forth at length.

Having now fully described this invention, it will be apparent to one of ordinary skill in the art that many changes and modifications can be made thereto without departing from the spirit or scope of the invention as set forth herein.

1. A method of protecting keratin materials from the harmful effects of pollution, comprising topically applying a composition comprising an effective amount of phytanetriol to said keratin materials.

2. The method of claim 1, wherein said effective amount ranges from 0.001 to 20% by weight, based on the weight of the composition.

3. The method of claim 1, wherein said effective amount ranges from 0.1% to 10% by weight, based on the weight of the composition.

4. The method of claim 1, wherein said composition is an emulsion.

5. The method of claim 1, wherein the phytanetriol is in the form of cubic gel particles.

6. The method of claim 5, wherein the cubic gel particles are in aqueous dispersion.

7. The method of claim 1, wherein the phytanetriol is in the form of cubic gel particles, and wherein said cubic gel particles are formed from a mixture comprising:
(i) 0.1% to 15% by weight, relative to the total weight of the composition, of phytanetriol or of a mixture of phytanetriol with a compound selected from the group consisting of N-2-alkoxycarbonyl derivatives of N-methylglucamine and unsaturated fatty acid monoglycerides; and
(ii) 0.05 to 3% by weight, relative to the total weight of the composition, of at least one dispersing and stabilizing agent, said agent being selected from the group consisting of surfactants that are water-soluble at room temperature and containing a saturated or unsaturated, linear or branched fatty chain containing from 8 to 22 carbon atoms.

8. The method of claim 7, wherein a weight proportion of compound (i) to said dispersing and stabilizing agent (ii) ranges from 2 to 200.

9. The method of claim 7, wherein said N-2-alkoxycarbonyl derivative of N-methylglucamine corresponds to formula (I) below: 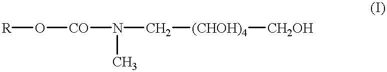
in which R represents a branched alkyl radical containing from 6 to 18 carbon atoms.

10. The method of claim 7, wherein said N-2-alkoxycarbonyl derivative of N-methylglucamine is selected from the group consisting of N-2-hexyldecyloxycarbonyl-N-methylglucamine, N-2-ethyl-hexyloxycarbonyl-N-methylglucamine and N-2-butyloctyloxycarbonyl-N-methylglucamine, and mixtures thereof.

11. The method of claim 7, wherein said cubic gel particles contain as compound (i) a mixture consisting of from 1% to 40% by weight of phytanetriol relative to the weight of the mixture and from 60% to 99% by weight of N-2-alkoxycarbonyl derivative of N-methylglucamine relative to the weight of the mixture.

12. The method of claim 7, wherein said unsaturated fatty acid monoglyceride is selected from the group consisting of glyceryl monooleate, glyceryl monolinoleate, and mixtures thereof.

13. The method of claim 7, wherein said cubic gel particles contain as compound (i) a mixture consisting of from 1% to 50% by weight of phytanetriol relative to the weight of the mixture and from 50% to 99% by weight of unsaturated fatty acid monoglyceride relative to the weight of the mixture.

14. The method of claim 7, wherein said said dispersing and stabilizing agent is selected from the group consisting of:
(1) alkyl or alkenyl ethers or esters of a polyol,
(2) N-acyl amino acids and derivatives thereof, and peptides N-acylated with an alkyl or alkenyl radical, and salts thereof,
(3) alkyl or alkenyl ether or ester sulphates, derivatives thereof and salts thereof,
(4) polyoxyethylenated fatty alkyl or alkenyl ethers or esters,
(5) polyoxyethylenated alkyl or alkenyl carboxylic acids and salts thereof,
(6) N-alkyl or alkenyl betaines,
(7) alkyl or alkenyl trimethylammoniums and salts thereof, and
(8) mixtures thereof.

15. The method of claim 5, wherein said cubic gel particles have a size ranging from 0.05 μm. to 1 μm.

16. The method of claim 7, wherein said cubic gel particles have a size ranging from 0.05 μm. to 1 μm.

17. The method of claim 6, wherein the dispersion of cubic gel particles further comprises at least one water-insoluble ionic amphiphilic lipid.

18. The method of claim 17, wherein said water-insoluble ionic amphiphilic lipid is at least one selected from the group consisting of:
(i) phospholipids,
(ii) phosphoric esters of fatty acids,
(iii) water-insoluble N-acyl derivatives of glutamic acid and salts thereof,
(iv) sodium cetyl sulphate,
(v) sodium cocoylmonoglyceride sulphate, and
(vi) water-insoluble quaternary ammonium derivatives.

19. The method of claim 5, wherein said cubic gel particles further comprise at least one hydrophilic and/or lipophilic active principle.

20. The method of claim 7, wherein said cubic gel particles further comprise at least one hydrophilic and/or lipophilic active principle.

21. The method of claim 5, wherein the cubic gel particles are present in an amount ranging from 0.1% to 20% by weight relative to the total weight of the composition.

22. The method of claim 7, wherein the cubic gel particles are present in an amount ranging from 0.1% to 20% by weight relative to the total weight of the composition.

23. A treatment process for protecting a keratin material against the effects of pollution, comprising applying to the keratin material a composition comprising an effective amount of phytanetriol in a physiologically acceptable medium.

24. A treatment process for improving the cell respiration and/or for reducing the desquamation of a keratin material and/or for preventing a keratin material from becoming dull and/or dirty and/or for preventing the dehydration of a keratin material, comprising applying to the keratin material a composition comprising an effective amount of phytanetriol in a physiologically acceptable medium.

25. A process for treating dry skin, comprising applying to the skin a composition comprising an effective amount of phytanetriol in a physiologically acceptable medium.

26. The process of claim 23, wherein said is an emulsion.

27. The process of claim 23, wherein the phytanetriol is in the form of cubic gel particles.

28. The process of claim 23, wherein said keratin material is the skin.

29. The process of claim 24, wherein said is an emulsion.

30. The process of claim 24, wherein the phytanetriol is in the form of cubic gel particles.

31. The process of claim 24, wherein said keratin material is the skin.

32. The process of claim 25, wherein said keratin material is the skin.

33. The process of claim 25, wherein said is an emulsion.

34. The process of claim 25, wherein the phytanetriol is in the form of cubic gel particles.

35. The process of claim 25, wherein said keratin material is the skin.

36. A composition, comprising:
phytanetriol in the form of cubic gel particles,
wherein said cubic gel particles are formed from a mixture comprising:
(i) 0.1% to 15% by weight, relative to the total weight of the composition, of phytanetriol or of a mixture of phytanetriol with a compound selected from the group consisting of N-2-alkoxycarbonyl derivatives of N-methylglucamine and unsaturated fatty acid monoglycerides; and
(ii) 0.05 to 3% by weight, relative to the total weight of the composition, of at least one dispersing and stabilizing agent, said agent being selected from the group consisting of surfactants that are water-soluble at room temperature and containing a saturated or unsaturated, linear or branched fatty chain containing from 8 to 22 carbon atoms.
